(19) United States
(12) Patent Application Publication
(10) Pub. No.: US 2023/0233110 A1
(43) Pub. Date: Jul. 27, 2023

(54) BLOOD MEASUREMENT DEVICE

(71) Applicants: LOOK TEC CO., LTD., Ota-shi, Gunma (JP); NATIONAL UNIVERSITY CORPORATION GUNMA UNIVERSITY, Maebashi-shi, Gunma (JP)

(72) Inventors: Hitoshi Ogura, Ota-shi, Gunma (JP); Koichi Takezawa, Ota-shi, Gunma (JP); Takayuki Asao, Maebashi-shi, Gunma (JP); Fumio Hayashi, Maebashi-shi, Gunma (JP)

(73) Assignees: LOOK TEC CO., LTD., Ota-shi, Gunma (JP); NATIONAL UNIVERSITY CORPORATION GUNMA UNIVERSITY, Maebashi-shi, Gunma (JP)

(21) Appl. No.: 17/928,587

(22) PCT Filed: Oct. 2, 2020
(86) PCT No.: PCT/JP2020/037616

§ 371 Date: Nov. 29, 2022

(51) Int. Cl. A61B 5/145; A61B 5/00

(52) U.S. Cl. CPC A61B 5/14532; A61B 5/0075

(57) ABSTRACT

Provided is a blood measurement device capable of accurately estimating the amount of a component contained in blood by passing light beams for calculating the amount of the component contained in blood along the same optical axis. A blood measurement device 10 of the present invention includes a light emitting part 11 having a first light emitting part 111 and a second light emitting part 112, a light receiving part 19, an actuator 16, and a computation and control part 17 that estimates a glucose level and controls operation of the actuator 16. When applicating a first light beam from the first light emitting part 111 to a measurement site, the computation and control part 17 causes the actuator 16 to move a light emission point of the first light emitting part 111 onto an optical axis 22 defined to penetrate through the measurement site, and when applicating a second light beam from the second light emitting part 112 to the measurement site, the computation and control part 17 causes the actuator 16 to move a light emission point of the second light emitting part 112 onto the optical axis 22.

### TECHNICAL FIELD

The present invention relates to a blood measurement device that optically measures the amount of a component contained in blood inside a measurement site of the human body or the like.

### BACKGROUND ART

There are an invasive approach and a non-invasive approach as methods for detecting sugar inside a measured site. In a method using an invasive approach, blood is drawn from, e.g., a fingertip of the human body, and the blood is used to measure a glucose level. In a method using a non-invasive approach, no blood is drawn from the human body, and a glucose level is measured with a sensor placed outside of the human body. While an invasive approach is common in order to calculate an accurate glucose level, a calculation device using a non-invasive approach is desired so as to mitigate pain and improve convenience for a user.

A known example of a device for measuring a glucose level using a non-invasive approach is one that performs optical measurement by applying near-infrared light or the like to the human body.

Also, as a device that optically measures a glucose level, there is one that detects a difference in an amount of near-infrared light absorbed by glucose. Specifically, this device causes near-infrared light to transmit through a given site and measures a glucose level from the amount of light transmitted (for example, Patent Literatures 1 and 2).

### CITATION LIST

### Patent Literature

Patent Literature 1: Japanese Patent No. 3093871
Patent Literature 2: Japanese Patent No. 3692751

### SUMMARY OF THE INVENTION

### Problem to be Solved by the Invention

However, the non-invasive glucose-level measurement device described in each of the above patent literatures has a problem of not necessarily being able to measure an accurate glucose level.

Specifically, the measurement technique described in Patent Literature 1 calculates a glucose level using a glucose oxidase method and thus has a problem of a glucose level calculation being complicated. Meanwhile, the measurement technique described in Patent Literature 2 measures a glucose level using an optical approach, but only to a level of being able to determine a possibility of diabetes, not to a level of being able to measure a glucose level quantitatively.

The present invention has been made in view of such problems and has an objective to provide a blood measurement device that can accurately estimate the amount of a component contained in blood by passing light beams for calculating the amount of the component contained in blood along the same optical axis.

### Means for Solving the Problem

A blood measurement device according to the present invention includes: a light emitting part having a first light emitting part that applies a first light beam of a first wavelength and a second light emitting part that applies a second light beam of a second wavelength; a light receiving part that receives the first light beam and the second light beam transmitting through a measurement site; an actuator that moves the light emitting part; and a computation and control part that estimates an amount of a component contained in blood based on light reception intensities of the first light beam and the second light beam and controls operation of the actuator. Here, when applicating the first light beam from the first light emitting part to the measurement site, the computation and control part causes the actuator to move a light emission point of the first light emitting part onto an optical axis defined to penetrate through the measurement site. When applicating the second light beam from the second light emitting part to the measurement site, the computation and control part causes the actuator to move a light emission point of the second light emitting part onto the optical axis.

In the blood measurement device according to the present invention, the measurement site is a finger web, and a sandwiching part to sandwich the finger web is formed outside the light emitting part and the light receiving part.

In the blood measurement device according to the present invention, the actuator brings the light emitting part or the light receiving part close to the finger web by moving the light emitting part or the light receiving part along the optical axis.

In the blood measurement device according to the present invention, the blood measurement device further includes a first pressing part and a second pressing part that are pressed against a site near the finger web when the finger web is inserted into the sandwiching part.

In the blood measurement device according to the present invention, an abutment part to abut against fingers sandwiching the finger web is formed outside the sandwiching part.

In the blood measurement device according to the present invention, the light emitting part further has a third light emitting part that applies a third light beam of a third wavelength, when applicating the third light beam from the third light emitting part to the measurement site, the computation and control part causes the actuator to move a light emission point of the third light emitting part onto the optical axis, and the computation and control part estimates the amount of the component contained in blood based on light reception intensities of the first light beam, the second light beam, and the third light beam.

In the blood measurement device according to the present invention, the blood measurement device further includes: a main body; a first waveguide and a second waveguide that protrude from the main body; and a first mirror and a second mirror provided in the first waveguide and the second waveguide, respectively. Here, the optical axis is defined to go through the first waveguide, the first mirror, the second waveguide, and the second mirror.

In the blood measurement device according to the present invention, the amount of the component contained in blood is a glucose level.

### Effect of the Invention

A blood measurement device according to the present invention includes: a light emitting part having a first light emitting part that applies a first light beam of a first wavelength and a second light emitting part that applies a second light beam of a second wavelength; a light receiving part that receives the first light beam and the second light beam transmitting through a measurement site; an actuator that moves the light emitting part; and a computation and control part that estimates an amount of a component contained in blood based on light reception intensities of the first light beam and the second light beam and controls operation of the actuator. Here, when applicating the first light beam from the first light emitting part to the measurement site, the computation and control part causes the actuator to move a light emission point of the first light emitting part onto an optical axis defined to penetrate through the measurement site. When applicating the second light beam from the second light emitting part to the measurement site, the computation and control part causes the actuator to move a light emission point of the second light emitting part onto the optical axis. According to such a blood measurement device of the present invention in which the first light beam and the second light beam are applied along the optical axis defined to penetrate through a measurement site, the optical paths along which the respective light beams pass and the lengths of the optical paths are unified. Owing to the optical conditions thus being uniformized between the first light beam and the second light beam, the amount of a component contained in blood can be measured accurately based on the light reception intensities of the first light beam and the second light beam transmitting through the measurement site.

In the blood measurement device according to the present invention, the measurement site is a finger web, and a sandwiching part to sandwich the finger web is formed outside the light emitting part and the light receiving part. According to such a blood measurement device of the present invention, by sandwiching their finger web with the sandwiching part, a user can make sure that the finger web is situated between the light emitting part and the light receiving part, which enables more accurate measurement of the amount of the component contained in blood. Also, a finger web has a short transmission distance and is therefore preferable as a measurement site.

In the blood measurement device according to the present invention, the actuator brings the light emitting part or the light receiving part close to the finger web by moving the light emitting part or the light receiving part along the optical axis. According to such a blood measurement device of the present invention in which the actuator brings the light emitting part or the light receiving part close to the finger web, the amount of the component contained in blood can be calculated more accurately.

In the blood measurement device according to the present invention, the blood measurement device further includes a first pressing part and a second pressing part that are pressed against a site near the finger web when the finger web is inserted into the sandwiching part. According to such a blood measurement device of the present invention in which the first pressing part and the second pressing part are pressed against muscle near the finger web, the finger web can have even more preferable positional relations to the light emitting part and the light receiving part at the time of application of the light beams.

In the blood measurement device according to the present invention, an abutment part to abut against fingers sandwiching the finger web is formed outside the sandwiching part. According to such a blood measurement device of the present invention, the finger web can be stretched to have an even thickness at the time of measurement.

In the blood measurement device according to the present invention, the light emitting part further has a third light emitting part that applies a third light beam of a third wavelength, when applicating the third light beam from the third light emitting part to the measurement site, the computation and control part causes the actuator to move a light emission point of the third light emitting part onto the optical axis, and the computation and control part estimates the amount of the component contained in blood based on light reception intensities of the first light beam, the second light beam, and the third light beam. According to such a blood measurement device of the present invention in which the amount of the component contained in blood is calculated using not only the light reception intensities of the first and second light beams but also that of the third light beam, the amount of the component contained in blood can be calculated more accurately.

In the blood measurement device according to the present invention, the blood measurement device further includes: a main body; a first waveguide and a second waveguide that protrude from the main body; and a first mirror and a second mirror provided in the first waveguide and the second waveguide, respectively. Here, the optical axis is defined to go through the first waveguide, the first mirror, the second waveguide, and the second mirror. According to such a blood measurement device of the present invention, the amount of the component contained in blood can be measured without the blood flow at the measurement site being compressed.

In the blood measurement device according to the present invention, the amount of the component contained in blood is a glucose level. According to such a blood measurement device of the present invention, a glucose level as the amount of the component contained in blood can be estimated accurately.

### BRIEF DESCRIPTION OF THE DRAWINGS

FIG. 1 is a diagram showing a blood measurement device according to an embodiment of the present invention, and FIGS. 1(A) and 1(B) are perspective views showing the blood measurement device.

FIG. 2 is a conceptual diagram showing a connection configuration of the blood measurement device according to the embodiment of the present invention.

FIG. 3 is a perspective view showing an actuator of the blood measurement device according to the embodiment of the present invention.

FIG. 4 is an exploded perspective view showing the actuator of the blood measurement device according to the embodiment of the present invention.

FIG. 5 shows a method for measuring a glucose level using the blood measurement device according to the embodiment of the present invention, and FIGS. 5(A) and 5(B) are top views sequentially showing how to apply the blood measurement device to a finger web.

FIG. 6 is a sectional view showing a method for detecting a glucose level using the blood measurement device according to the embodiment of the present invention and showing how the blood measurement device is applied to a finger web.

FIG. 7 is a diagram showing a glucose level calculation method according to the embodiment of the present invention, and FIGS. 7(A), 7(B), 7(C), and 7(D) are side views showing how measurement is performed while moving the light emitting point and bringing a light emitting part and a light receiving part close to each other.

FIG. 8 is a diagram showing a glucose level calculation method according to the embodiment of the present invention, FIG. 8(A) being a schematic diagram showing a finger web, FIG. 8(B) being a graph showing results of glucose level measurement using a fingertip, and FIG. 8(C) being a graph showing results of glucose level measurement using a finger web.

FIG. 9 is a sectional view showing a method for detecting a glucose level using a blood measurement device according to a different mode of the present invention and showing how the blood measurement device is applied to a finger web.

### MODES FOR CARRYING OUT THE INVENTION

A blood measurement device 10 according to an embodiment of the present invention is described in detail below based on the drawings. In the following description, the same reference number is basically used for the same members to omit repetitive descriptions. In the present embodiment, a glucose level is employed as an example of the amount of a component contained in blood measured by the blood measurement device 10.

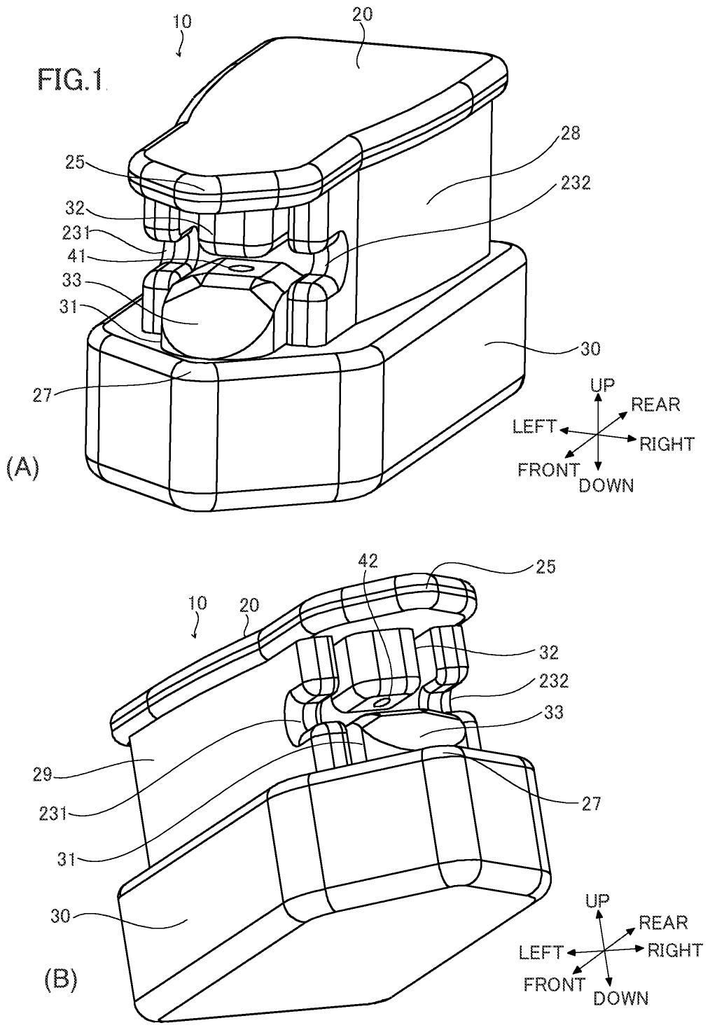

With reference to FIG. 1, the outer appearance and the like of the blood measurement device 10 of the present mode are described. FIG. 1(A) is a perspective view of the blood measurement device 10 seen from the upper front, and FIG. 1(B) is a perspective view of the blood measurement device 10 seen from the lower front.

Referring to FIGS. 1(A) and 1(B), the design part of the blood measurement device 10 is formed of, e.g., a synthetic resin. Also, the blood measurement device 10 as a whole has a substantially cuboid shape whose longitudinal direction is along the front-rear direction. When the blood measurement device 10 is seen from above, a center part of a front end is protruding frontward. The blood measurement device 10 has a size and weight that allow a user who measures a glucose level with the blood measurement device 10 to hold the blood measurement device 10 with one hand. Here, a glucose level is a blood or interstitial glucose level. Also, a glucose level is sometimes referred to as a blood sugar level or the like.

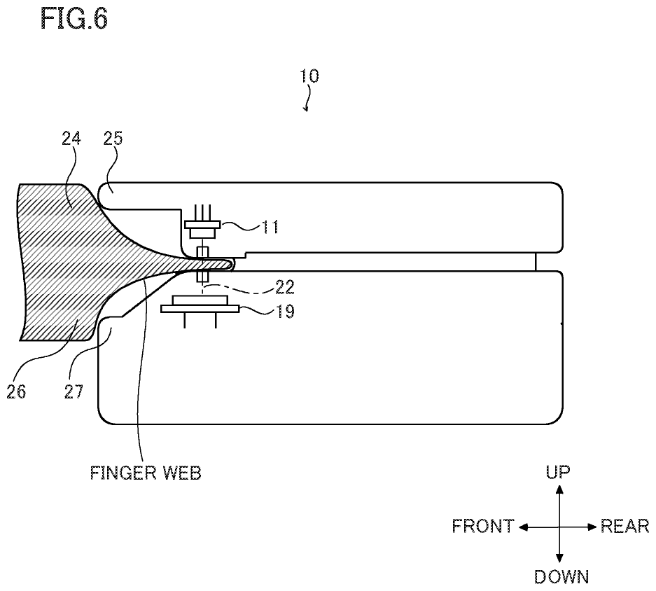

An actuator housing part 30 is formed at a lower portion of the blood measurement device 10. A mechanism for changing the position of a light emitting part 11 to be described later is housed in the actuator housing part 30, the configuration of which will be described later. An area around a center portion of the front surface of the actuator housing part 30 protrudes frontward, thereby forming a second pressing part 27. The second pressing part 27 is pressed against a particular body site of the human body when the blood measurement device 10 is used to measure a glucose level, and details will be described later with reference to FIG. 6.

An upper plate part 20 is formed at an upper end portion of the blood measurement device 10. An area near a center part of the front face of the upper plate part 20 protrudes frontward, thereby forming a first pressing part 25. The first pressing part 25 is pressed against a particular site of the human body when the blood measurement device 10 is used to measure a glucose level, and will be described in detail later with reference to FIG. 6.

Referring to FIG. 1(A), an abutment part 28 is formed at an upper portion of the right side surface of the blood measurement device 10. The abutment part 28 may be a flat surface or a curved surface depressing inward to allow a user's finger to fit therein. The abutment part 28 is a part against which, for example, a user's thumb abuts when the blood measurement device 10 is used to calculate a glucose level.

A front end portion of the abutment part 28 is partially notched to from a sandwiching part 232. The sandwiching part 232 is wide enough in the up-down direction to allow insertion of a finger web to be described later. Also, a rear end of the sandwiching part 232 is situated more rearward than an opening part 41 through which light beams for measurement pass. Thus, bringing the peripheral portion of a finger web, which is a measurement site, into abutment against the rear end of the sandwiching part 232 ensures that the finger web is situated at the opening part 41 and thus ensures that the light beams passing through the opening part 41 transmit through the finger web.

Referring to FIG. 1(B), an abutment part 29 is formed at an upper portion of the left side surface of the blood measurement device 10. The abutment part 29 may be a flat surface or a curved surface depressing inward to allow a user's finger to fit therein. The abutment part 29 is a part against which, for example, a user's index finger abuts when the blood measurement device 10 is used to calculate a glucose level.

A front end portion of the abutment part 29 is partially notched to form a sandwiching part 231. The specific shape of the sandwiching part 231 is the same as that of the sandwiching part 232 described above.

Referring to FIG. 1(A), a light-emitting-part housing part 31 is formed near a front end of the upper surface of the actuator housing part 30. The light-emitting-part housing part 31 is provided between the sandwiching part 231 and the sandwiching part 232 in the left-right direction. The light-emitting-part housing part 31 is a part in which the light emitting part 11 to be described later is situated. Also, a slanted surface 33 is formed at a front portion of the light-emitting-part housing part 31, slanting downward to the front. Forming the slanted surface 33 allows a finger web to be guided into the sandwiching part 231 and the sandwiching part 232 along the slanted surface 33 at the time of glucose level measurement. Also, an upper surface of the light-emitting-part housing part 31 is opened to form the opening part 41.

Referring to FIG. 1(B), a light-receiving-part housing part 32 is formed, protruding downward from a front portion of the lower surface of the upper plate part 20. The light-receiving-part housing part 32 is a part to house a light receiving part 19 to be described later. Also, an opening part 42 is formed at the lower surface of the light-receiving-part housing part 32 to allow light beams used for glucose level measurement to pass therethrough.

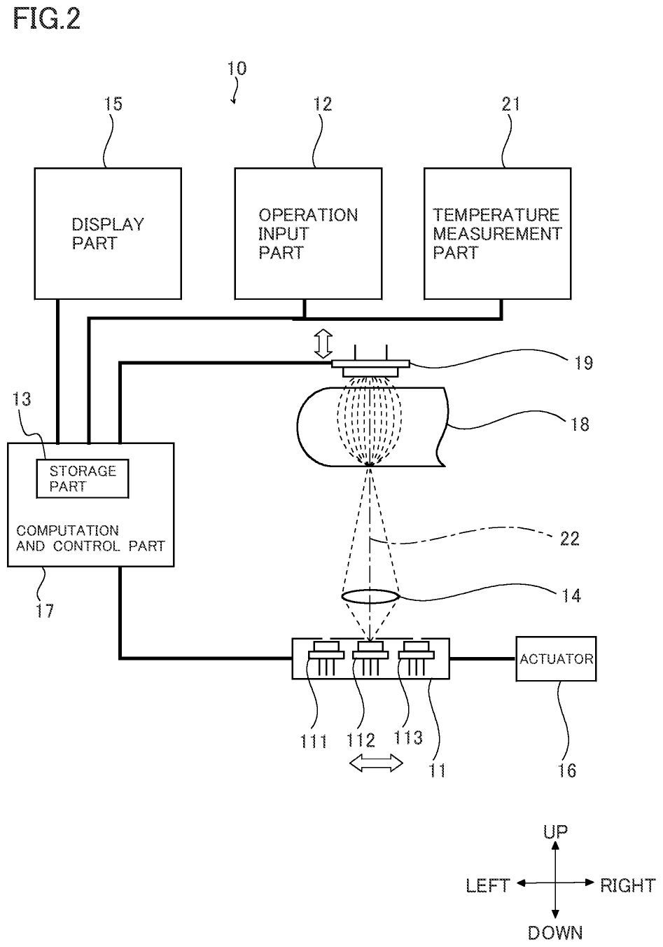

FIG. 2 is a conceptual diagram showing a basic configuration of the blood measurement device 10. Referring to FIG. 2, the blood measurement device 10 has the light emitting part 11 that emits light beams used for measurement, a lens 14 which is an optical element that leads a light beam emitted from the light emitting part 11 to a measurement site 18, the light receiving part 19 that receives a light beam transmitting through the measurement site 18, a computation and control part 17 that calculates a glucose level based on an output from the light receiving part 19, a storage part 13, a display part 15, an operation input part 12, and a temperature measurement part 21. Here, a pinhole can be used in place of the lens 14 to focus a light beam.

The function of the blood measurement device 10 is to measure a glucose level in the human body using a non-invasive approach by causing light beams to transmit through the human body which is a measurement site.

The light emitting part 11 emits light beams of predetermined wavelengths in order to measure a glucose level. The light emitting part 11 has a first light emitting part 111, a second light emitting part 112, and a third light emitting part 113 that emit light beams of different wavelengths. The first light emitting part 111, the second light emitting part 112, and the third light emitting part 113 are each made of a light emission diode. For example, the wavelength of a first light beam emitted from the first light emitting part 111 is 1310 nm, the wavelength of a second light beam emitted from the second light emitting part 112 is 1450 nm, and the wavelength of a third light beam emitted from the third light emitting part 113 is 1550 nm.

The first light beam is a light beam not absorbed by a component in living organisms, while the second and third light beams are light beams absorbed by glucose, protein, and water in living organisms. The first light beam is used to measure the optical path length of an optical axis 22 in order to measure the influence of the optical path length on the absorbency of each light beam and eliminate the influence of the optical path length, which enables accurate calculation of a glucose level.

An actuator 16 moves the light emitting part 11 in the left-right direction. Moving the light emitting part 11 allows the light emission point of one of the first light emitting part 111, the second light emitting part 112, and the third light emitting part 113 to be situated on the same optical axis 22. In the case shown here, the light emission point of the second light emitting part 112 is situated on the optical axis 22.

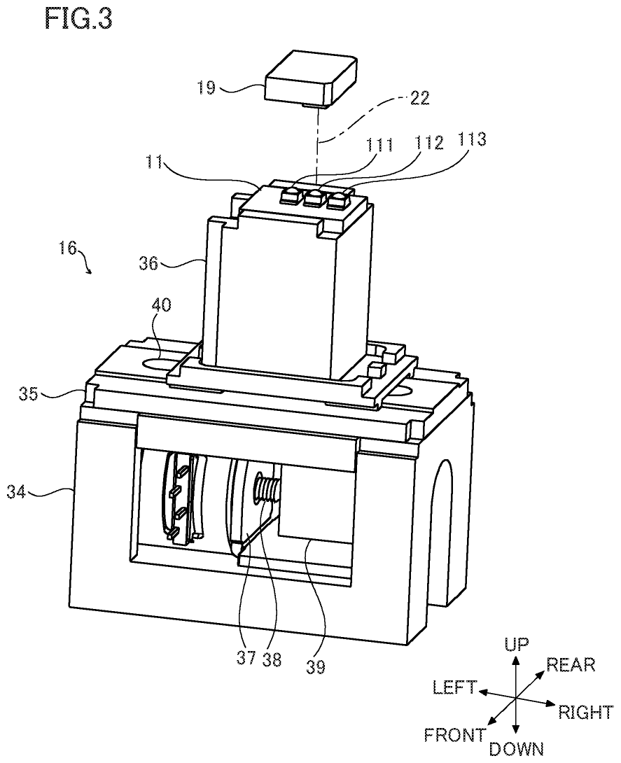

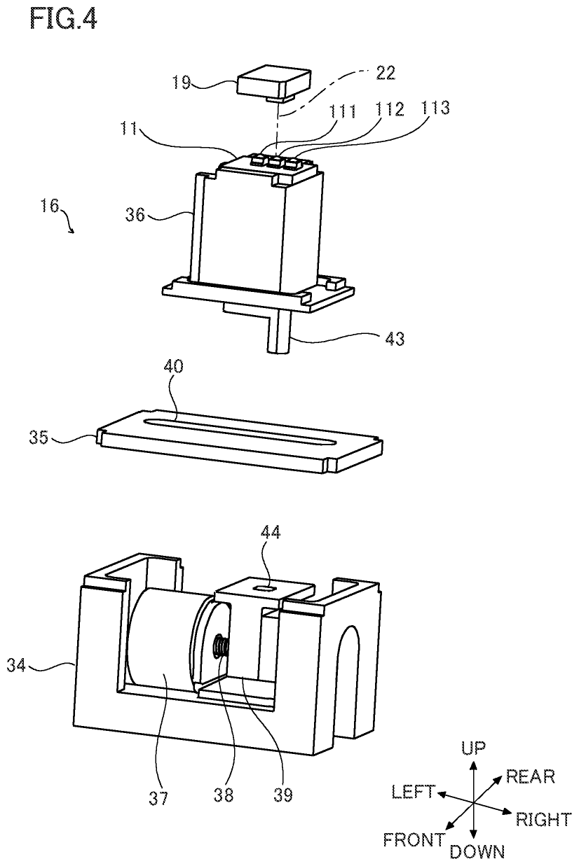

The actuator 16 also moves one or both of the light emitting part 11 and the light receiving part 19 in the up-down direction. For example, the actuator 16 brings the light receiving part 19 away from the light emitting part 11 in time of receiving measurement where the blood measurement device 10 is not measuring a glucose level. Meanwhile, the actuator 16 brings the light receiving part 19 and the light emitting part 11 close to each other in time of measurement where the blood measurement device 10 measures a glucose level. An example of a specific configuration of the actuator 16 will be described later with reference to FIGS. 3 and 4.

In the present embodiment, the first light beam, the second light beam, and the third light beam are applied from the light emitting part 11 to the light receiving part 19 along the same optical axis 22. In other words, the first light beam, the second light beam, and the third light beam have the same propagation path and propagation length inside the measurement site 18.

Because the light beams share the optical axis 22 as described above, a glucose level can be measured accurately. Specifically, according to the Lambert-Beer law, a glucose level is calculated using the following Formula 1:

C=−log₁₀(I/I₀)/(0.434×μₐ×r) Formula 1

In Formula 1 above, C is a glucose level, I is the power of emitted light, I₀ is the power of incident light, μₐ is the light absorption coefficient of the skin, and r is an optical path length.

In the present embodiment, the first light beam, the second light beam, and the third light beam have the same optical path length r by sharing the optical axis 22, and thus, there are fewer unknown numbers to be calculated, which makes it possible to find the glucose level C accurately and easily.

The lens 14 guides the first light beam, the second light beam, and the third light beam emitted from the first light emitting part 111, the second light emitting part 112, and the third light emitting part 113 described above, respectively, to the measurement site 18 using its refraction action and diffraction action.

The measurement site 18 is a site for which a glucose level is measured by the blood measurement device 10 of the present mode. Specifically, a fingertip, an earlobe, a finger web, or the like can be used as the measurement site 18. As will be described later, a finger web is preferable as the measurement site 18 because a finger web contains only a small amount of fat, does not vary in its thickness much between individuals, and has no thick vein formed therein.

The light receiving part 19 is a semiconductor device made of, for example, a photodiode and has a light receiving part formed to receive the first light beam, the second light beam, and the third light beam transmitting through the measurement site 18 and detect the intensity of the light received. The light receiving part 19 sends the computation and control part 17 signals according to the light reception intensities of the first light beam, the second light beam, and the third light beam.

The storage part 13 is, e.g., a semiconductor storage device made of a RAM or a ROM, and stores calculation formulae for calculating a glucose level from an output value from the light receiving part 19, parameters, estimation results, programs for executing a glucose level calculation method, and the like.

The operation input part 12 is a part with which a user gives instructions to the computation and control part 17 and is formed of a switch, a touch panel, or the like.

The temperature measurement part 21 is a part that measures the body temperature of a user when brought into contact with the body of the user.

The computation and control part 17, which is formed of a CPU, performs various kinds of computation and also controls the operation of the parts forming the blood measurement device 10. To be more specific, the computation and control part 17 applies the first light beam, the second light beam, and the third light beam from the first light emitting part 111, the second light emitting part 112, and the third light emitting part 113 of the light emitting part 11. Also, the computation and control part 17 estimates a glucose level using a conversion formula based on electrical signals inputted from, e.g., the light receiving part 19 and the temperature measurement part 21. Also, the computation and control part 17 may display a calculated glucose level on the display part 15. When the glucose level is displayed on the display part 15, which is, for example, a liquid crystal display, a user using the blood measurement device 10 can know a change in their glucose level in real time. Further, the computation and control part 17 operates the actuator 16 to move the light emitting part 11 and the light receiving part 19 in order to situate the light emission point of each light emitting part in the shape of the optical axis 22 at the time of measurement.

The actuator 16 is described in detail with reference to FIGS. 3 and 4. FIG. 3 is a perspective view showing the actuator 16, and FIG. 4 is an exploded perspective view showing the actuator 16 exploded in the up-down direction.

Referring to FIG. 3, the actuator 16 primarily has a housing 34, a lid part 35, a holder 36, a motor 37, a rotary shaft 38, and a threaded engagement part 39. The actuator 16 situates the light emission point of the first light emitting part 111, the second light emitting part 112, or the third light emitting part 113 on the optical axis 22 by moving the light emitting part 11 in the left-right direction based on an instruction from the computation and control part 17 described above. Note that the light reception point of the light receiving part 19 is also situated on the optical axis 22.

A specific operation of the actuator 16 is as follows. Based on an instruction from the computation and control part 17 described above, the motor 37 rotates the rotary shaft 38, causing the threaded engagement part 39 in either threaded engagement or engagement with the rotary shaft 38 to move in the left-right direction. When the threaded engagement part 39 moves in the left-right direction, the holder 36 having the light emitting part 11 placed on top also moves in the left-right direction together. Consequently, the light emission point of the first light emitting part 111, the second light emitting part 112, or the third light emitting part 113 can be situated in the shape of the optical axis 22.

The parts forming the actuator 16 are described with reference to FIG. 4. The housing 34 is a substantially box-shaped part having an open top. The motor 37, the rotary shaft 38, and the threaded engagement part 39 are placed in the housing 34.

Inside the housing 34, part of the rotary shaft 38 extending from the motor 37 is situated in the threaded engagement part 39. A thread groove is formed on the side surface of the rotary shaft 38. As the rotary shaft 38 rotates, the threaded engagement part 39 moves in the left-right direction by coming into threaded engagement or engagement with the thread groove on the rotary shaft 38. A hole part 44 is formed in the upper surface of the threaded engagement part 39. The hole part 44 is situated below an opening part 40 to be described later.

The lid part 35 is a plate-shaped member closing the opening in the upper surface of the housing 34. The opening part 40, which is elongated in the left-right direction, is formed in the lid part 35.

The holder 36 has a substantially cuboid shape and has the light emitting part 11 provided on the upper surface thereof. Also, a substantially bar-shaped protrusion part 43 is formed at the holder 36, protruding downward. The protrusion part 43 passes through the opening part 40 and is inserted into the hole part 44.

Because the actuator 16 is configured as described above, when the motor 37 rotates the rotary shaft 38 in one direction based on an instruction from the computation and control part 17, the rotation causes the threaded engagement part 39 to move rightward, which causes the holder 36 and the light emitting part 11 to move rightward. Conversely, when the motor 37 rotates the rotary shaft 38 in the opposite direction based on an instruction from the computation and control part 17, the rotation causes the threaded engagement part 39 to move leftward, which causes the holder 36 and the light emitting part 11 to move leftward. Thus configured, the actuator 16 can situate the light emission point of one of the first light emitting part 111, the second light emitting part 112, and the third light emitting part 113 in the shape of the same optical axis 22. Meanwhile, in the front-rear and left-right directions, the light receiving part 19 is fixed in its position in the front-rear and left-right directions at all times and is situated in the shape of the optical axis 22.

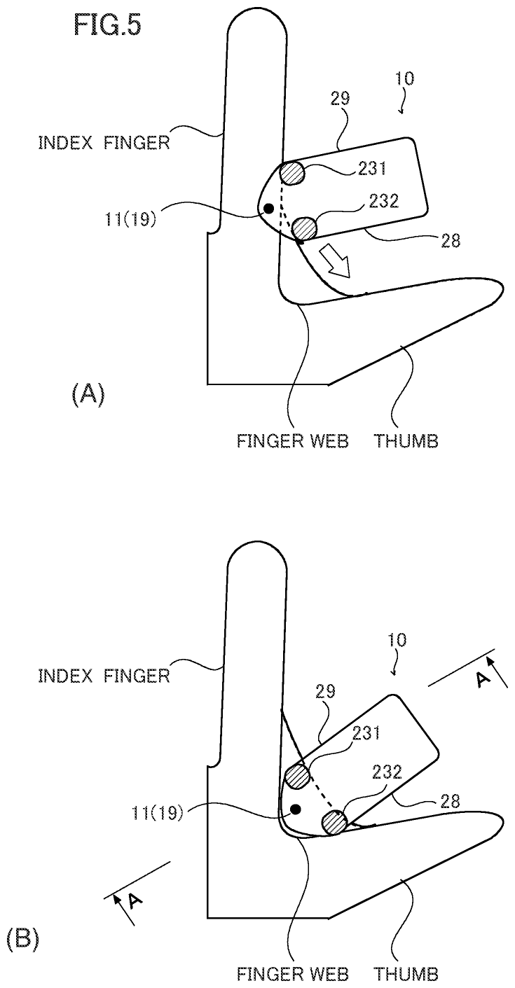

Next, based on FIGS. 5 to 7 and also with reference to FIGS. 1 to 4 above, a description is given of a specific method for measuring a glucose level of a user by using the blood measurement device 10 configured as described above.

Referring to FIG. 5, first, the blood measurement device 10 is set at a user's measurement site. FIGS. 5A and 5B are diagrams sequentially showing how the blood measurement device 10 is set at a finger web as a measurement site. A finger web formed between the thumb and index finger of the left hand is employed here as a site for measuring a glucose level. Thus, a user operates the blood measurement device 10 with their right hand easily.

Referring to FIG. 5(A), the blood measurement device 10 is fitted from a part of the finger web on the index finger side. Specifically, the sandwiching part 232 and the sandwiching part 231 of the blood measurement device 10 are slid from the part of the finger web on the index finger side. In this stage, the user is stretching the finger web by spreading the thumb and the index finger.

Referring to FIG. 5(B), next, with the finger web still being sandwiched between the sandwiching part 231 and the sandwiching part 232, the blood measurement device 10 is moved toward the thumb side while being pressed leftward. Here, the blood measurement device 10 is slid until the tip end portion of the blood measurement device 10 reaches the base of the thumb or the vicinities thereof.

An edge portion of the finger web is now in abutment with the rear ends of the sandwiching part 231 and the sandwiching part 232. This allows the light emitting part 11 and the light receiving part 19 to be situated at parts overlapping with the finger web. In this state, each light beam emitted from the light emitting part 11 transmits through the finger web and reaches the light receiving part 19.

Here, the base of the thumb or the vicinities thereof may be brought into contact with the abutment part 28 of the blood measurement device 10. Also, the base of the index finger or the vicinities thereof may be brought into contact with the abutment part 29 of the blood measurement device 10. This allows the thumb and the finger index to spread by a certain angle or more, thereby preventing the finger web from sagging. This also allows the thumb and the finger index to spread by an even angle during glucose level measurement, thereby allowing a finger web thickness to be constant.

FIG. 6 is a sectional view taken along the cutting plane line A-A in FIG. 5(B). Referring to FIG. 6, the first pressing part 25 of the blood measurement device 10 is pressing the hand's adductor pollicis muscle 24 or the vicinities thereof frontward. Also, the second pressing part 27 of the blood measurement device 10 is pressing a ball 26 of the mother and child and the vicinities thereof frontward. This allows the relative positions between the finger web and the light emitting part 11 and the light receiving part 19 to have a predetermined positional relation, thereby allowing accurate calculation of a glucose level using light beams transmitting through the finger web.

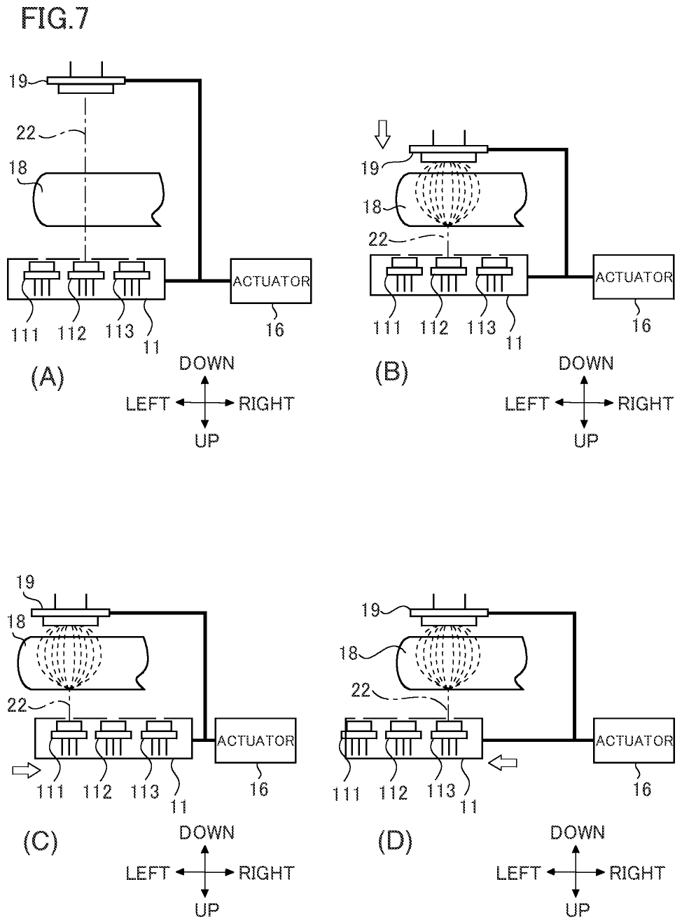

With reference to FIG. 7, a description is given of how each light beam is applied while changing the position of the light emitting part 11. FIG. 7(A) shows the light emitting part 11 before light beam application, FIG. 7(B) shows how the second light beam is applied from the second light emitting part 112, FIG. 7(C) shows how the first light beam is applied from the first light emitting part 111, and FIG. 7(D) shows how the third light beam is applied from the third light emitting part 113. Although the light beams are applied along the optical axis 22 in the order of the second light emitting part 112, the first light emitting part 111, and the third light emitting part 113 here, the order can be changed.

Referring to FIG. 7(A), the light emitting part 11 and the light receiving part 19 are situated to sandwich the measurement site 18, which is a finger web, in the up-down direction. The actuator 16 shortens the distance between the light emitting part 11 and the light receiving part 19 in the up-down direction by moving one or both of the light emitting part 11 and the light receiving part 19 in the up-down direction.

Here, based on an instruction from the computation and control part 17 described above, the actuator 16 moves the light receiving part 19 downward. For example, referring to FIG. 1(A), moving the light-receiving-part housing part 32 downward can lower the light receiving part 19 placed in the light-receiving-part housing part 32. Here, the finger web may or may not be sandwiched between the light-emitting-part housing part 31 and the light-receiving-part housing part 32 so as to have a constant thickness.

Referring to FIG. 7(B), to apply the second light beam from the second light emitting part 112, the computation and control part 17 first causes the actuator 16 to move the light emitting part 11 so that the light emission point of the second light emitting part 112 may be superimposed over the optical axis 22. Once the light emission point of the second light emitting part 112 is superimposed over the optical axis 22, the computation and control part 17 emits a second light beam from the second light emitting part 112. The emitted second light beam travels along the optical axis 22, transmits through the measurement site 18, and is then applied to the light receiving part 19. An electric signal indicating the intensity of the second light beam received by the light receiving part 19 is sent to the computation and control part 17.

Referring to FIG. 7(C), next, the computation and control part 17 causes the actuator 16 to move the light emitting part 11 rightward so that the light emission point of the first light emitting part 111 may be superimposed over the optical axis 22. Once the light emission point of the first light emitting part 111 is superimposed over the optical axis 22, the computation and control part 17 emits a first light beam from the first light emitting part 111. The emitted first light beam travels along the optical axis 22, transmits through the measurement site 18, and is then applied to the light receiving part 19. An electric signal indicating the intensity of the first light beam received by the light receiving part 19 is sent to the computation and control part 17.

Referring to FIG. 7(D), next, the computation and control part 17 causes the actuator 16 to move the light emitting part 11 leftward so that the light emission point of the third light emitting part 113 may be superimposed over the optical axis 22. Once the light emission point of the third light emitting part 113 overlaps with the optical axis 22, the computation and control part 17 emits a third light beam from the third light emitting part 113. The emitted third light beam travels along the optical axis 22, transmits through the measurement site 18, and is then applied to the light receiving part 19. An electric signal indicating the intensity of the third light beam received by the light receiving part 19 is sent to the computation and control part 17.

Meanwhile, based on an instruction from the computation and control part 17, the temperature measurement part 21 measures the body temperature of the user, and an electric signal indicating the body temperature is sent to the computation and control part 17.

After the reception intensities of the first light beam, the second light beam, and the third light beam are measured using the above method, a glucose level of the user is calculated based on, e.g., the reception intensities of the light beams and the body temperature. For example, a statistical method can be used as this calculation method. As an example, a multi-regression curve is created by statistical analysis using glucose levels in blood drawn from users, the reception intensities of light beams, body temperatures, and the like. Then, using the regression curve, an estimated glucose level is calculated from the reception intensities of the light beams and the body temperature.

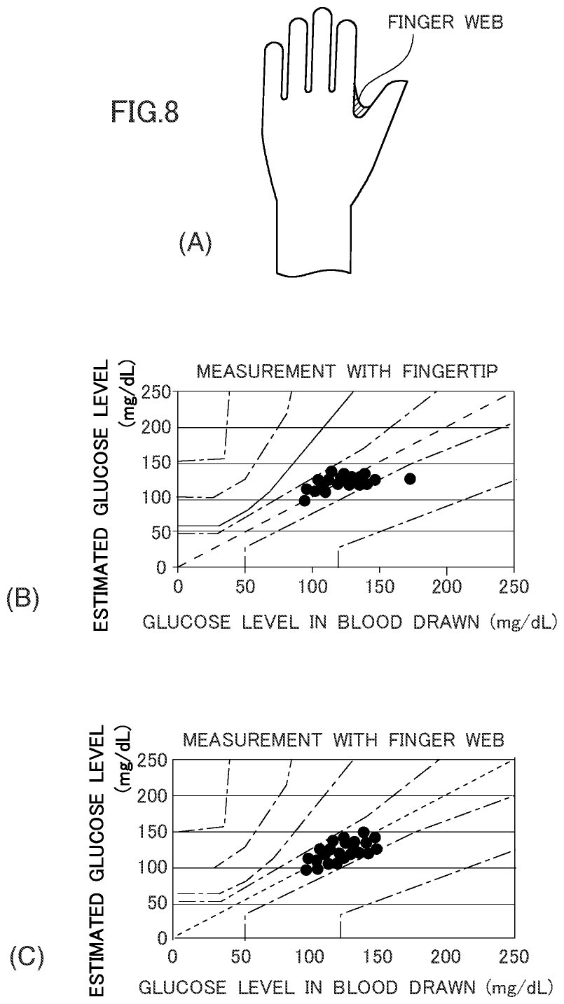

With reference to FIG. 8, a description is given of why a finger web is preferable as a measurement site to which light beams are applied to estimate a glucose level. FIG. 8(A) is a schematic diagram showing a user's hand, FIG. 8(B) is a graph showing an error grid for a case of glucose level estimation using a fingertip, and FIG. 8(C) is a graph showing an error grid for a case of glucose level estimation using a finger web. In FIGS. 8B and 8C, the horizontal axis represents a glucose level in blood drawn, and the vertical axis represents an estimated glucose level measured by a method according to the present embodiment.

Referring to FIG. 8(A), a finger web is a film-like part formed between fingers of the human body. Here, a finger web formed between other fingers can also be used as a measurement site.

Referring to FIG. 8(B), dots indicative of measurement results are distributed away from a reference line indicated by a broken line. Conceivable reasons for this are because the thickness of a fingertip varies greatly between individuals, which makes the optical path length different, and because a thick vein present in a fingertip has an adverse influence.

Meanwhile, referring to FIG. 8(C), dots indicative of measurement results are distributed near the reference line indicated by a broken line. This is because a finger web is approximately 2 mm to 4 mm in thickness and does not vary much between individuals, contains an extremely small amount of fat, and has no thick vein inside, which enables measurement using the capillary vessels and dermis. Further, when a finger web is used as a measurement site, the optical path length can be shortened, allowing a glucose level to be measured with low-output light.

Table 1 is referred to to describe why a finger web is preferable as a measurement site, from the perspective of a fat content.

TABLE 1

| | Sample 1 | Sample 2 | |
|---|---|---|---|
| | Epidermis 0.2 mm | Epidermis 0.2 mm | |
| | Dermis 0.8 mm | Dermis 0.8 mm | ratio |
| Wavelength (nm) | Fat 1.5 mm | No fat | (times) |
| 1310 | 5.40% | 18.50% | 3.4 |
| (First light beam) | | | |
| 1450 | 0.31% | 1.91% | 6.2 |
| (Second light beam) | | | |
| 1550 | 0.22% | 0.77% | 3.5 |
| (Third light beam) | | | |

Table 1 shows measurement results of transmittances of a sample 1 containing fat (epidermis 0.2 mm, dermis 0.8 mm, fat 1.5 mm) and a body donation 2 containing no fat (epidermis 0.2 mm, dermis 0.8 mm, no fat) for the first light beam, the second light beam, and the third light beam. As an example, the sample 1 is a human's fingertip, which contains fat, and the sample 2 is a finger web.

The simulation conditions here are as follows: the number of light beams is 5000, the number of times of diffusion per light beam is 1000, the diameter of light incident on the skin is φ1.5 mm, and the diameter of the light receiving plane is φ3 mm or φ1 mm.

As shown in Table 1, for the first light beam having a wavelength of 1310 nm, the transmittance of the sample 2 is 3.4 times that of the sample 1. Also, for the second light beam having a wavelength of 1450 nm, the transmittance of the sample 2 is 6.2 times that of the sample 1. Further, for the third light beam having a wavelength of 1550 nm, the transmittance of the sample 2 is 3.5 times that of the sample 1.

Judging from the above, the sample 1, which is a fingertip as an example, is not preferable as a site for measuring a glucose level because of the low transmittances for the first and third light beams. Further, considering that a fat content varies greatly between individuals, it is obvious that the quantity of fat affects the transmittance, making it difficult to estimate a glucose level.

By contrast, the sample 2, which is a finger web and contains an extremely small amount of fat, transmits the first light beam, the second light beam, and the third light beam well, making it possible to accurately estimate a glucose level based on the intensities of the transmitted light beams. Also, even if a user is obese, fat contained in a finger web does not increase extremely. Thus, when a glucose level is estimated using light beams transmitting through a finger web, the glucose level can be estimated accurately without being affected by whether the user is obese.

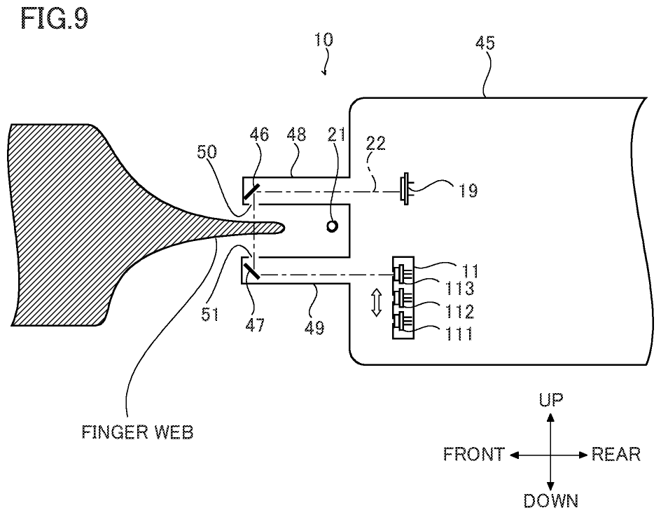

The configuration of a blood measurement device 10 according to a different mode is described with reference to FIG. 9. FIG. 9 is a sectional view of the blood measurement device 10 according to the difference mode. The basic configuration of the blood measurement device 10 shown in FIG. 9 is the same as that shown in FIGS. 1 to 4. Thus, the following omits descriptions of the same portions of the configuration and method, focusing mainly on different portions.

The blood measurement device 10 has a main body 45, and constituent members forming the blood measurement device 10 are placed in the main body 45. Here, the light emitting part 11 and the light receiving part 19 placed in the main body 45 are shown.

A waveguide 48 and a waveguide 49 protrude from the front end surface of the main body 45, with a mirror 46 placed in the waveguide 48 and a mirror 47 placed in the waveguide 49. The light receiving part 19 is situated rearward of the waveguide 48, and the light emitting part 11 is situated rearward of the waveguide 49. Further, a portion of the waveguide 48 below the mirror 46 is opened to form an opening 50, and a portion of the waveguide 49 above the mirror 47 is opened to form an opening 51. Further, the mirror 46 and the mirror 47 are situated at the front ends of the waveguide 48 and the waveguide 49. Also, the temperature measurement part 21 is situated between the waveguide 48 and the waveguide 49.

Formed inside the main body 45 is the optical axis 22 along which each light beam passes at the time of glucose level measurement. The optical axis 22 is defined to go through the light emitting part 11, the waveguide 49, the mirror 47, the opening 51, the opening 50, the mirror 46, the waveguide 48, and the light receiving part 19.

To calculate a glucose level using the blood measurement device 10, first, a finger web is situated between the waveguide 48 and the waveguide 49. Consequently, the finger web is positioned between the opening 50 and the opening 51. Further, the temperature measurement part 21 comes into contact with the finger web and measures the body temperature.

Next, the computation and control part 17 applies each light beam from the light emitting part 11 along the optical axis 22. Each light beam emitted from the light emitting part 11 passes through the waveguide 49, gets reflected by the mirror 47, passes through the opening 51, transmits through the finger web, passes through the opening 50, gets reflected by the mirror 46, passes through the waveguide 48, and reaches the light reception point of the light receiving part 19. The computation and control part 17 moves the light emitting part 11 in the up-down directions to emit the first light beam, the second light beam, and the third light beam from the emission points of the first light emitting part 111, the second light emitting part 112, and the third light emitting part 113, respectively, along the optical axis 22. Here, the waveguide 48 and the waveguide 49 are either away from each other not to compress the finger web or lightly in contact with the finger web.

Next, the light receiving part 19 sends the computation and control part 17 an electric signal indicating the light reception intensity of each light beam. The computation and control part 17 calculates a glucose level using a conversion formula such as a multi-regression equation and using, e.g., the light reception intensities of the light beams and the temperature of the finger web measured by the temperature measurement part 21.

In the blood measurement device 10 shown in FIG. 9, the waveguide 48 and the waveguide 49 are not pressed against the finger web or a site in the vicinities thereof. Thus, a glucose level can be calculated accurately with good blood flow inside the finger web.

Although embodiments of the present invention have thus been shown, the present invention is not limited to the embodiments above.

For example, although the first light beam, the second light beam, and the third light beam of different wavelengths are used to calculate a glucose level in the present embodiments described above, two light beams (e.g., the first light beam of a wavelength of 1310 nm and the third light beam of a wavelength of 1550 nm) can also be used to calculate a glucose level.

Also, the blood measurement device 10 described above can also be used for purposes other than glucose level measurement. For example, diseases such as cancer may be diagnosable from the intensities of the light beams received by the light receiving part 19 after transmitting through a human body.

Further, although a finger formed between the thumb and the index finger is used as a measurement site in the present embodiments described above, a finger web formed between other fingers can also be used as a measurement site.

### REFERENCE SIGNS LIST

10 blood measurement device
11 light emitting part
111 first light emitting part
112 second light emitting part
113 third light emitting part
12 operation input part
13 storage part
14 lens
15 display part
16 actuator
17 computation and control part
18 measurement site
19 light receiving part
20 upper plate part
21 temperature measurement part
22 optical axis
231 sandwiching part
232 sandwiching part
24 adductor pollicis muscle
25 first pressing part
26 ball of the mother and child
27 second pressing part
28 abutment part
29 abutment part
30 actuator housing part
31 light-emitting-part housing part
32 light-receiving-part housing part
33 slanted surface
34 housing
35 lid part
36 holder
37 motor
38 rotary shaft
39 threaded engagement part
40 opening part
41 opening part
42 opening part
43 protrusion part
44 hole part
45 main body
46 mirror
47 mirror
48 waveguide
49 waveguide
50 opening
51 opening

1-8. (canceled)

9. A blood measurement device comprising:
a light emitting part having a first light emitting part that applies a first light beam of a first wavelength and a second light emitting part that applies a second light beam of a second wavelength;
a light receiving part that receives the first light beam and the second light beam transmitting through a measurement site;
an actuator that moves the light emitting part; and
a computation and control part that estimates an amount of a component contained in blood based on light reception intensities of the first light beam and the second light beam and controls operation of the actuator, wherein
when applicating the first light beam from the first light emitting part to the measurement site, the computation and control part causes the actuator to move a light emission point of the first light emitting part onto an optical axis defined to penetrate through the measurement site,
when applicating the second light beam from the second light emitting part to the measurement site, the computation and control part causes the actuator to move a light emission point of the second light emitting part onto the optical axis,
the measurement site is a finger web, and
a sandwiching part to sandwich the finger web is formed outside the light emitting part and the light receiving part.

10. The blood measurement device according to claim 9, wherein
the actuator brings the light emitting part or the light receiving part close to the finger web by moving the light emitting part or the light receiving part along the optical axis.

11. The blood measurement device according to claim 9, comprising a first pressing part and a second pressing part that are pressed against a site near the finger web when the finger web is inserted into the sandwiching part.

12. The blood measurement device according to claim 9, wherein
an abutment part to abut against fingers sandwiching the finger web is formed outside the sandwiching part.

13. The blood measurement device according to claim 9, wherein
the light emitting part further has a third light emitting part that applies a third light beam of a third wavelength,
when applicating the third light beam from the third light emitting part to the measurement site, the computation and control part causes the actuator to move a light emission point of the third light emitting part onto the optical axis, and
the computation and control part estimates the amount of the component contained in blood based on light reception intensities of the first light beam, the second light beam, and the third light beam.

14. The blood measurement device according to claim 9, further comprising:
a main body;
a first waveguide and a second waveguide that protrude from the main body; and
a first mirror and a second mirror provided in the first waveguide and the second waveguide, respectively, wherein
the optical axis is defined to go through the first waveguide, the first mirror, the second waveguide, and the second mirror.

15. The blood measurement device according to claim 9, wherein the amount of the component contained in blood is a glucose level.
